(19) United States
(12) Patent Application Publication
(10) Pub. No.: US 2025/0352149 A1
(43) Pub. Date: Nov. 20, 2025

(54) APPARATUS AND METHOD FOR LEFT VENTRICULAR EJECTION FRACTION PREDICTION

(71) Applicant: Anumana, Inc., Cambridge, MA (US)

(72) Inventor: Samir Awasthi, Boston, MA (US)

(73) Assignee: Anumana, Inc., Cambridge, MA (US)

(21) Appl. No.: 18/666,363

(22) Filed: May 16, 2024

(51) Int. Cl. A61B 5/00; A61B 5/28; A61B 5/308; A61B 5/364; G16H 10/60; G16H 50/20; G16H 50/30

(52) U.S. Cl. CPC A61B 5/7275; A61B 5/28; A61B 5/308; A61B 5/364; A61B 5/742; G16H 10/60; G16H 50/20; G16H 50/30

(57) ABSTRACT

Described herein is an apparatus and a method for left ventricular ejection fraction (LVEF) prediction. An apparatus may include at least a processor, and a memory communicatively connected to the at least processor, wherein the memory contains instructions configuring the at least processor to receive a first electrocardiogram (ECG) datum; input the first ECG datum into an LVEF prediction model; and receive as an output from the LVEF prediction model a first LVEF prediction.

### FIELD OF THE INVENTION

The present invention generally relates to the field of machine learning. In particular, the present invention is directed to an apparatus and method for left ventricular ejection fraction prediction.

### BACKGROUND

Left ventricular ejection fraction is typically determined using echocardiograms. However, these often require a specialist to perform an echocardiogram procedure and interpret the results. Electrocardiograms are non-invasive, and the necessary equipment, or data from past electrocardiograms, is often available.

### SUMMARY OF THE DISCLOSURE

In an aspect, an apparatus for left ventricular ejection fraction (LVEF) prediction may include at least a processor; and a memory communicatively connected to the at least processor, wherein the memory contains instructions configuring the at least processor to receive a first electrocardiogram (ECG) datum; input the first ECG datum into an LVEF prediction model; and receive as an output from the LVEF prediction model a first LVEF prediction wherein the first LVEF prediction comprises a confidence metric.

In another aspect, a method of left ventricular ejection fraction (LVEF) prediction, may include, using at least a processor, receiving a first electrocardiogram (ECG) datum; using the at least a processor, inputting the first ECG datum into an LVEF prediction model; and using the at least a processor, receiving as an output from the LVEF prediction model a first LVEF prediction, wherein the first LVEF prediction comprises a confidence metric.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a diagram depicting an exemplary embodiment of an apparatus for left ventricular ejection fraction prediction;

FIG. 2 is a box diagram of an exemplary machine learning model;

FIG. 3 is a diagram of an exemplary neural network;

FIG. 4 is a diagram of an exemplary neural network node;

FIG. 5 is a flow diagram depicting an exemplary embodiment of a method of left ventricular ejection fraction prediction;

FIG. 6 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to systems and methods for left ventricular ejection fraction (LVEF) prediction. In some embodiments, an ECG sensor may be used to collect an ECG datum. In some embodiments, such ECG datum may be input into an LVEF prediction model, and the model may output an LVEF prediction. This LVEF prediction may be used to determine one or more variables associated with cardiac conditions of the subject. The LVEF prediction and/or variable determined based on the prediction may be displayed to a user, such as a doctor or a subject.

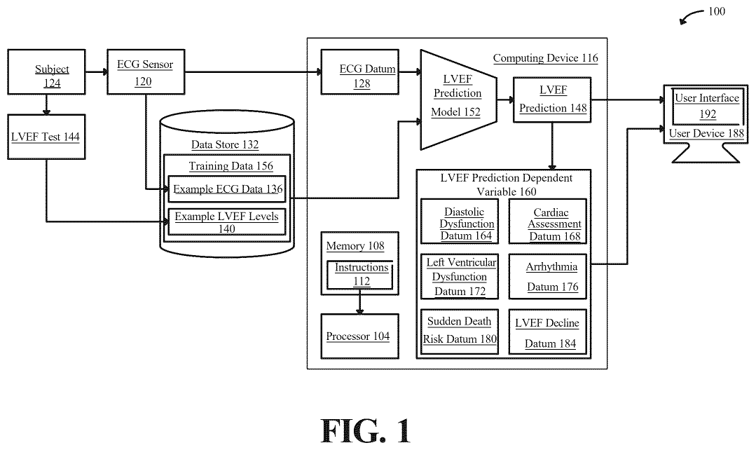

Referring now to FIG. 1, an exemplary embodiment of an apparatus 100 for LVEF prediction is illustrated. Apparatus 100 may include a computing device. Apparatus 100 may include a processor. Processor may include, without limitation, any processor described in this disclosure. Processor may be included in computing device. Computing device may include any computing device as described in this disclosure, including without limitation a microcontroller, microprocessor, digital signal processor (DSP) and/or system on a chip (SoC) as described in this disclosure. Computing device may include, be included in, and/or communicate with a mobile device such as a mobile telephone or smartphone. Computing device may include a single computing device operating independently, or may include two or more computing device operating in concert, in parallel, sequentially or the like; two or more computing devices may be included together in a single computing device or in two or more computing devices. Computing device may interface or communicate with one or more additional devices as described below in further detail via a network interface device. Network interface device may be utilized for connecting computing device to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device.

Still referring to FIG. 1, in some embodiments, apparatus 100 may include at least a processor 104 and a memory 108 communicatively connected to the at least a processor 104, the memory 108 containing instructions 112 configuring the at least a processor 104 to perform one or more processes described herein. Computing device 116 may include processor 104 and/or memory 108. Computing device 116 may be configured to perform one or more processes described herein.

Still referring to FIG. 1, computing device 116 may include but is not limited to, for example, a computing device or cluster of computing devices in a first location and a second computing device or cluster of computing devices in a second location. Computing device 116 may include one or more computing devices dedicated to data storage, security, distribution of traffic for load balancing, and the like. Computing device 116 may distribute one or more computing tasks as described below across a plurality of computing devices of computing device, which may operate in parallel, in series, redundantly, or in any other manner used for distribution of tasks or memory between computing devices. Computing device 116 may be implemented, as a non-limiting example, using a “shared nothing” architecture.

With continued reference to FIG. 1, computing device 116 may be designed and/or configured to perform any method, method step, or sequence of method steps in any embodiment described in this disclosure, in any order and with any degree of repetition. For instance, computing device 116 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Computing device 116 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Still referring to FIG. 1, as used in this disclosure, “communicatively connected” means connected by way of a connection, attachment or linkage between two or more relata which allows for reception and/or transmittance of information therebetween. For example, and without limitation, this connection may be wired or wireless, direct or indirect, and between two or more components, circuits, devices, systems, and the like, which allows for reception and/or transmittance of data and/or signal(s) therebetween. Data and/or signals therebetween may include, without limitation, electrical, electromagnetic, magnetic, video, audio, radio and microwave data and/or signals, combinations thereof, and the like, among others. A communicative connection may be achieved, for example and without limitation, through wired or wireless electronic, digital or analog, communication, either directly or by way of one or more intervening devices or components. Further, communicative connection may include electrically coupling or connecting at least an output of one device, component, or circuit to at least an input of another device, component, or circuit. For example, and without limitation, via a bus or other facility for intercommunication between elements of a computing device. Communicative connecting may also include indirect connections via, for example and without limitation, wireless connection, radio communication, low power wide area network, optical communication, magnetic, capacitive, or optical coupling, and the like. In some instances, the terminology “communicatively coupled” may be used in place of communicatively connected in this disclosure.

Still referring to FIG. 1, in some embodiments, apparatus 100 may include an electrocardiogram (ECG) sensor 120. ECG sensor 120 may include one or more electrodes. Electrodes may be placed on subject 124 such as on chest, arms, and legs of subject 124. Electrodes may detect electrical impulses produced by the heart. Lead wires may be used to connect electrodes to a computing device of an ECG sensor. ECG sensor 120 may receive electrical signals from electrodes, may amplify such signals and convert them into a visual representation, such as a waveform. ECG sensor 120 may include one or more lead wires. ECG sensor 120 may include a device configured to measure and/or interpret electrical activity of heart of subject 124 using electrodes and/or lead wires. In some embodiments, ECG sensor 120 may be configured to detect ECG datum 128 and/or transmit ECG datum 128 to computing device 116. As used herein, an “ECG datum” is a datum describing electrical activity of the heart of a subject. In some embodiments, an ECG datum may include a rhythm strip ECG datum. As used herein, a “rhythm strip ECG datum” is a datum describing electrical activity detected using a single electrode. In some embodiments, an ECG datum may include a median beat ECG datum. As used herein, a “median beat ECG datum” is a datum describing electrical activity detected using a plurality of leads and/or electrodes. In some embodiments, ECG datum 128 may include data collected by 2, 3, 4, 5, 6, 7, 8, 9, 10, 11, 12, or more ECG leads. For example, ECG datum 128 may include a median beat collected by 12 ECG leads. In some embodiments, an ECG datum may be associated with a particular subject 124. In some embodiments, subject 124 may have a ventricular pacemaker.

Still referring to FIG. 1, in some embodiments, ECG datum 128 may be stored in a data store 132 and/or memory 108. Data store 132 may be implemented, without limitation, as a relational database, a key-value retrieval database such as a NOSQL database, or any other format or structure for use as a database that a person skilled in the art would recognize as suitable upon review of the entirety of this disclosure. Data store 132 may alternatively or additionally be implemented using a distributed data storage protocol and/or data structure, such as a distributed hash table or the like. Data store 132 may include a plurality of data entries and/or records as described above. Data entries in a data store 132 may be flagged with or linked to one or more additional elements of information, which may be reflected in data entry cells and/or in linked tables such as tables related by one or more indices in a relational database. Persons skilled in the art, upon reviewing the entirety of this disclosure, will be aware of various ways in which data entries in a database may store, retrieve, organize, and/or reflect data and/or records as used herein, as well as categories and/or populations of data consistently with this disclosure. ECG data stored in data store 132 may be used as example ECG data 136 for training a machine learning model as described below. In some embodiments, data store 132 may include an electronic health record database. In some embodiments, an electronic health record database may include health information such as ECG data and example LVEF levels from a plurality of subjects. In some embodiments, health information may be received in an anonymized state and/or may be anonymized by apparatus 100, such as by removing identifying information.

Still referring to FIG. 1, in some embodiments, computing device 116 may receive ECG datum 128. In some embodiments, computing device 116 may receive ECG datum 128 from ECG sensor 120. In some embodiments, computing device 116 may receive ECG datum 128 from data store 132. For example, computing device 116 may receive ECG datum 128 from data store 132 in a situation in which a subject previously received an ECG and now wishes to know the LVEF of a subject and/or an aspect of cardiac health dependent on LVEF of the subject.

Still referring to FIG. 1, in some embodiments, data store 132 and/or computing device 116 may receive example LVEF levels 140. In some embodiments, subject 124 may receive LVEF test 144. In some embodiments, apparatus 100 may include an LVEF test sensor. A LVEF test 144 may include, in non-limiting examples, an echocardiogram (such as a transthoracic echocardiogram), resonance imaging (MRI) techniques, computerized tomography (CT) techniques, and/or nuclear medicine scans. In some embodiments, example LVEF levels 140 may be used to train a machine learning model as described herein.

Still referring to FIG. 1, in some embodiments, apparatus 100 may determine LVEF prediction 148. As used herein, an “LVEF prediction” is a data structure encapsulating an estimate of the LVEF of a subject, where the estimate is a point on a number line, a range determined based on a point on a number line, or both. LVEF is the percent of the blood in the left ventricle of the heart that is ejected from the left ventricle during a contraction of the heart. Normally, LVEF is above 50%. Lower LVEF can indicate that a subject has or is likely to have significant heart problems. An LVEF prediction may include, for example, an estimate that a subject's LVEF is a particular value, such as 45.1%. In another example, an LVEF prediction may include an estimate that a subject's LVEF is within a margin or confidence interval of a particular value, such as 48.6%+1%. However, an estimate that a subject's LVEF is in a predetermined value range is not an LVEF prediction. For example, an estimate that a subject's LVEF is in a predetermined category of 40%-50% is not an LVEF prediction. Similarly, an estimate that a subject's LVEF is in a predetermined category of above 50% is not an LVEF prediction. In some embodiments, LVEF prediction 148 is determined based on ECG datum 128. In some embodiments, LVEF prediction 148 is not determined based on another measurement of a subject's heart, such as an echocardiogram.

Still referring to FIG. 1, in some embodiments, LVEF prediction model 152 may be used to determine LVEF prediction 148. LVEF prediction model 152 may be trained using a supervised learning algorithm. LVEF prediction model 152 may include a regression model. LVEF prediction model 152 may include a neural network. LVEF prediction model 152 may be trained on training data 156 including example ECG data 136, associated with example LVEF levels 140. In some embodiments, training data 156 may include ECG features associated with LVEF levels 140. Such training dataset may be obtained by, for example, receiving training data 156 from data store 132. Example ECG data 136 may include ECG data obtained from electrocardiograms performed on prior subjects. In some embodiments, example ECG data 136 may be supplemented with ECG datum 128 associated with subject 124. In some embodiments, such data may be used to modify and/or retrain LVEF prediction model 152. Example LVEF levels 140 may be obtained from prior LVEF tests such as echocardiograms and may be supplemented with data from an LVEF test applied to subject 124, such as for model retraining purposes. Once LVEF prediction model 152 is trained, it may be used to determine LVEF prediction 148. Apparatus 100 may input ECG datum 128 into LVEF prediction model 152, and apparatus 100 may receive LVEF prediction 148 from the model.

With continued reference to FIG. 1, LVEF prediction 148 may include a confidence metric. A “confidence metric,” for the purposes of this disclosure, is one or more datum concerning the probability of an event. In some embodiments, confidence metric may include a confidence score. Confidence score may reflect the predicted accuracy of LVEF prediction 148. Confidence score may be on a range from 0 to 1, 0 to 10, 0 to 5, 0 to 100, or the like. A confidence score closer to the upper bound of the range (e.g., 1) may reflect higher confidence, whereas a confidence score closer to the lower bound of the range (e.g., 0) may reflect lower confidence. In some embodiments, a confidence metric may include a confidence interval. A confidence interval, for the purposes of this disclosure is an interval that is expected to contain the parameter being estimated to a given confidence level. In some embodiments, confidence metric may include a margin or error.

Still referring to FIG. 1, in some embodiments, example ECG data 136 and/or ECG datum 128 may include data in the form of a data structure with dimensions including a number of leads of an ECG and a number of sequentially captured readings. In a non-limiting example, data captured from a 12-lead ECG monitored at 500 Hz for 10 seconds may include 5000 data points per lead and may produce an input ECG shape of 5000×12×1. In some embodiments, ECG data with different frequency, duration, and/or number of leads may be used. In some embodiments, ECGs may be distributed into training, validation, and test data sets. Such distribution may be random. In some embodiments, a training dataset may include 70% of an overall dataset, a validation dataset may include 10% of an overall dataset, and a test dataset may include 20% of an overall dataset.

Still referring to FIG. 1, in some embodiments, apparatus 100 may identify one or more LVEF prediction dependent variables 160 as a function of LVEF prediction 148. As used herein, an “LVEF prediction dependent variable” is a datum determined as a function of an LVEF prediction. LVEF prediction dependent variable 160 may include, in non-limiting examples, a diastolic dysfunction datum 164, a cardiac assessment datum 168, a left ventricular dysfunction datum 172, an arrhythmia datum 176, a sudden death risk datum 180, and/or an LVEF decline datum 184. In some embodiments, LVEF prediction dependent variable 160 may be determined as a function of a comparison between LVEF prediction 148 and a threshold. As used herein, an LVEF prediction which includes a range is “above” a threshold if the center point of the range is greater than the threshold. As used herein, an LVEF prediction which includes a range is “below” a threshold if the center point of the range is less than the threshold.

Still referring to FIG. 1, in some embodiments, apparatus 100 may determine diastolic dysfunction datum 164. In some embodiments, apparatus 100 may determine diastolic dysfunction datum 164 as a function of LVEF prediction 148. As used herein, a “diastolic dysfunction datum” is a datum indicating that a subject has diastolic dysfunction, has an increased likelihood of diastolic dysfunction, or both. In some embodiments, apparatus 100 may determine diastolic dysfunction datum 164 if LVEF prediction 148 is above 40%, 45%, 50%, 55%, 60%, 65%, or 70%. For example, diastolic dysfunction datum 164 may be determined by comparing LVEF prediction 148 to such a value. In some embodiments, apparatus 100 may determine diastolic dysfunction datum 164 if LVEF prediction 148 is above 50%. In some embodiments, diastolic dysfunction datum 164 may be categorical. In a non-limiting example, a first category of diastolic dysfunction datum 164 associated with having diastolic dysfunction may be determined if LVEF prediction 148 is above 50%, and a second category of diastolic dysfunction datum 164 associated with not having diastolic dysfunction may be determined if LVEF prediction 148 is below 50%.

Still referring to FIG. 1, in some embodiments, apparatus 100 may determine cardiac assessment datum 168. In some embodiments, apparatus 100 may determine cardiac assessment datum 168 as a function of LVEF prediction 148. As used herein, a “cardiac assessment datum” is a datum indicating that a subject needs, or may need, an assessment of cardiac health. In some embodiments, apparatus 100 may determine cardiac assessment datum 168 if LVEF prediction 148 is below 20%, 25%, 30%, 35%, 40%, 45%, or 50%. For example, cardiac assessment datum 168 may be determined by comparing LVEF prediction 148 to such a value. In some embodiments, apparatus 100 may determine cardiac assessment datum 168 if LVEF prediction 148 is below 45%. In some embodiments, cardiac assessment datum 168 may be categorical. In a non-limiting example, a first category of cardiac assessment datum 168 associated with needing an assessment of cardiac health may be determined if LVEF prediction 148 is below 45%, and a second category of cardiac assessment datum 168 associated with not needing an assessment of cardiac health may be determined if LVEF prediction 148 is above 45%.

Still referring to FIG. 1, in some embodiments, apparatus 100 may left ventricular dysfunction datum 172. In some embodiments, apparatus 100 may determine may left ventricular dysfunction datum 172 as a function of LVEF prediction 148. As used herein, a “may left ventricular dysfunction datum” is a datum indicating that a subject has left ventricular dysfunction, has an increased likelihood of left ventricular dysfunction, or both. In some embodiments, apparatus 100 may determine may left ventricular dysfunction datum 172 if LVEF prediction 148 is below 20%, 25%, 30%, 35%, 40%, 45%, or 50%. For example, left ventricular dysfunction datum 172 may be determined by comparing LVEF prediction 148 to such a value. In some embodiments, apparatus 100 may determine cardiac assessment datum 168 if LVEF prediction 148 is below 50%. In some embodiments, apparatus 100 may determine left ventricular dysfunction datum 172 indicating that subject 124 has, or may have, mild left ventricular dysfunction if LVEF prediction is below 50%. In some embodiments, apparatus 100 may determine left ventricular dysfunction datum 172 if LVEF prediction 148 is below 40%. In some embodiments, apparatus 100 may determine left ventricular dysfunction datum 172 indicating that subject 124 has, or may have, moderate left ventricular dysfunction if LVEF prediction is below 40%. In some embodiments, apparatus 100 may determine left ventricular dysfunction datum 172 if LVEF prediction 148 is below 30%. In some embodiments, apparatus 100 may determine left ventricular dysfunction datum 172 indicating that subject 124 has, or may have, severe left ventricular dysfunction if LVEF prediction is below 30%. In some embodiments, left ventricular dysfunction datum 172 may be categorical. In a non-limiting example, a first category of left ventricular dysfunction datum 172 associated with no left ventricular dysfunction may be determined if LVEF prediction 148 is above 50% %, a second category of left ventricular dysfunction datum 172 associated with mild left ventricular dysfunction may be determined if LVEF prediction 148 is between 40% and 50%, a third category of left ventricular dysfunction datum 172 associated with moderate left ventricular dysfunction may be determined if LVEF prediction 148 is between 30% and 40%, a fourth category of left ventricular dysfunction datum 172 associated with severe left ventricular dysfunction may be determined if LVEF prediction 148 is below 30%.

Still referring to FIG. 1, in some embodiments, apparatus 100 may determine arrhythmia datum 176. In some embodiments, apparatus 100 may determine arrhythmia datum 176 as a function of LVEF prediction 148. As used herein, an “arrhythmia datum” is a datum indicating that a subject has an arrhythmia, has an increased likelihood of arrhythmia, or both. In some embodiments, apparatus 100 may determine arrhythmia datum 176 if LVEF prediction 148 is below 20%, 25%, 30%, 35%, 40%, 45%, or 50%. For example, arrhythmia datum 176 may be determined by comparing LVEF prediction 148 to such a value. In some embodiments, apparatus 100 may determine arrhythmia datum 176 if LVEF prediction 148 is below 35%. In some embodiments, arrhythmia datum 176 may be categorical. In a non-limiting example, a first category of arrhythmia datum 176 associated with having an arrhythmia may be determined if LVEF prediction 148 is below 35%, and a second category of arrhythmia datum 176 associated with not having an arrhythmia may be determined if LVEF prediction 148 is above 35%.

Still referring to FIG. 1, in some embodiments, apparatus 100 may determine sudden death risk datum 180. In some embodiments, apparatus 100 may determine sudden death risk datum 180 as a function of LVEF prediction 148. As used herein, a “sudden death risk datum” is a datum indicating that a subject has an elevated risk of sudden death, may develop an elevated risk of sudden death, or both. In some embodiments, apparatus 100 may determine sudden death risk datum 180 if LVEF prediction 148 is below 20%, 25%, 30%, 35%, 40%, 45%, or 50%. For example, sudden death risk datum 180 may be determined by comparing LVEF prediction 148 to such a value. In some embodiments, apparatus 100 may determine sudden death risk datum 180 if LVEF prediction 148 is below 35%. In some embodiments, sudden death risk datum 180 may be categorical. In a non-limiting example, a first category of sudden death risk datum 180 associated with having a low risk of sudden death may be determined if LVEF prediction 148 is above 35%, and a second category of sudden death risk datum 180 associated with having a higher risk of sudden death may be determined if LVEF prediction 148 is below 35%.

Still referring to FIG. 1, in some embodiments, LVEF prediction 148 may be used to determine LVEF decline datum 184. As used herein, an “LVEF decline datum” is a datum indicating that the LVEF of a subject has decreased between a first measurement of the subject's LVEF and a second measurement of the subject's LVEF. A first LVEF prediction generated using LVEF prediction model 152, and/or second LVEF prediction generated using LVEF prediction model 152 may be used to determine LVEF decline datum 184. In some embodiments, an LVEF prediction not generated using LVEF prediction model 152 may be used to determine LVEF decline datum 184. For example, a first LVEF prediction may be made using an echocardiogram, a second LVEF prediction may be made using LVEF prediction model 152, and LVEF decline datum 184 may be determined as a function of such LVEF predictions. In some embodiments, LVEF decline datum 184 may be categorical. In a non-limiting example, a first category of LVEF decline datum 184 associated with having no drop in LVEF may be determined if a difference in LVEF predictions is above 0%, a second category of LVEF decline datum 184 associated with having a minor drop in LVEF may be determined if a difference in LVEF predictions is below 5%, a third category of LVEF decline datum 184 associated with having a moderate drop in LVEF may be determined if a difference in LVEF predictions is between 5% and 10%, and a fourth category of LVEF decline datum 184 associated with having a major drop in LVEF may be determined if a difference in LVEF predictions is above 10%.

Still referring to FIG. 1, in some embodiments, apparatus 100 may receive a first ECG datum and a second ECG datum, input the first ECG datum and the second ECG datum into LVEF prediction model 152, receive as an output from LVEF prediction model 152 a first LVEF prediction and a second LVEF prediction, and determine LVEF decline datum 184 as a function of such first LVEF prediction and second LVEF prediction, wherein the second ECG datum is recorded after the first ECG datum, the first LVEF prediction is determined as a function of the first ECG datum, and the second LVEF prediction is determined as a function of the second ECG datum. For example, apparatus 100 may determine LVEF decline datum 184 if such second LVEF prediction is at least 0.5%, 1%, 2%, 3%, 4%, 5%, 6%, 7%, 8%, 9%, 10%, 11%, 12%, 13%, 14%, 15%, or more lower than such first LVEF prediction. For example, apparatus 100 may determine LVEF decline datum 184 if such second LVEF prediction is at least 5% lower than such first LVEF prediction. For example, apparatus 100 may determine LVEF decline datum 184 if such second LVEF prediction is at least 10% lower than such first LVEF prediction.

Still referring to FIG. 1, in some embodiments, apparatus 100 may determine whether there has been an increase in LVEF of a subject. For example, apparatus 100 may determine whether there has been an increase in LVEF of 0.5%, 1%, 2%, 3%, 4%, 5%, 6%, 7%, 8%, 9%, 10%, 11%, 12%, 13%, 14%, 15%, or more between a first LVEF prediction and a second LVEF prediction.

Still referring to FIG. 1, in some embodiments, apparatus 100 may identify when a subject with heart failure decompensates. In some embodiments, identification of when a subject decompensates may include identifying elevated filling pressures and/or myocardial strain. In some embodiments, identification of myocardial strain may include identification of elevated levels of N-terminal pro b-type natriuretic peptide (NT-proBNP). In some embodiments, this may be implemented using a natriuretic peptide test. In some embodiments, an LVEF prediction may be used to estimate elevated filling pressures and/or myocardial strain.

Still referring to FIG. 1, in some embodiments, apparatus 100 may identify a cardiac condition such as left ventricular dysfunction in a subject. In some embodiments, identification of a cardiac condition such as left ventricular dysfunction in a subject may be done as a function of LVEF prediction 148. In some embodiments, a therapy may be administered to a subject as a function of LVEF prediction 148. For example, a therapy approved to treat a cardiac condition such as left ventricular dysfunction may be administered to a subject with LVEF prediction 148 indicating that the subject is likely to have a cardiac condition such as left ventricular dysfunction. In some embodiments, an appointment with a medical professional may be made based on LVEF prediction 148. For example, an appointment with a medical professional may be made for subjects with LVEF prediction 148 indicating that they are likely to have a cardiac condition such as left ventricular dysfunction. Such an appointment may be used to, for example, discuss treatment options.

Still referring to FIG. 1, in some embodiments, apparatus 100 may display a datum described herein to a user, such as subject 124 and/or a medical professional. As examples, apparatus 100 may display LVEF prediction 148, diastolic dysfunction datum 164, cardiac assessment datum 168, left ventricular dysfunction datum 172, arrhythmia datum 176, sudden death risk datum 180, and/or LVEF decline datum 184 to a user. User device 188 and/or user interface 192 may be used to display a datum. In some embodiments, apparatus 100 may transmit a signal including LVEF prediction 148 to user device 188, and the signal may configure user device 188 to communicate a datum to a user. User device 188 may communicate a datum to a user using, for example, a visual or audio format. Apparatus 100 may communicate a visual element and/or visual element data structure including a datum to user device 188. This may configure user device 188 to display a visual element, such as by using user interface 192 to do so. As used herein, a device “displays” a datum if the device outputs the datum in a format suitable for communication to a user. For example, a device may display a datum by outputting text or an image on a screen or outputting a sound using a speaker.

Still referring to FIG. 1, in some embodiments, a visual element data structure may include a visual element. As used herein, a “visual element” is a datum that is displayed visually to a user. As used herein, a “visual element” is a datum that is displayed visually to a user. In some embodiments, a visual element data structure may include a rule for displaying visual element. In some embodiments, a visual element data structure may be determined as a function of a datum such as LVEF prediction 148. In some embodiments, a visual element data structure may be determined as a function of an item from the list consisting of LVEF prediction 148, diastolic dysfunction datum 164, cardiac assessment datum 168, left ventricular dysfunction datum 172, arrhythmia datum 176, sudden death risk datum 180, and LVEF decline datum 184. In a non-limiting example, a visual element data structure may be generated such that visual element describing or highlighting a datum such as LVEF prediction 148 is displayed to a user such as a doctor or subject 124. In another example, a visual element may.

Still referring to FIG. 1, in some embodiments, visual element may include one or more elements of text, images, shapes, charts, particle effects, interactable features, and the like. For example, a visual element of an LVEF prediction may provide its value and a color coded background indicating the degree to which that value is healthy.

Still referring to FIG. 1, a visual element data structure may include rules governing if or when visual element is displayed. In a non-limiting example, a visual element data structure may include a rule causing a visual element describing a datum such as LVEF prediction 148 to be displayed when a user selects a datum such as LVEF prediction 148 using a graphical user interface (GUI).

Still referring to FIG. 1, a visual element data structure may include rules for presenting more than one visual element, or more than one visual element at a time. In an embodiment, about 1, 2, 3, 4, 5, 10, 20, or 50 visual elements are displayed simultaneously.

Still referring to FIG. 1, a visual element data structure rule may apply to a single visual element or datum, or to more than one visual element or datum. For example, a visual element data structure may rank visual elements and/or other data and/or apply numerical values to them, and a computing device may display a visual element as a function of such rankings and/or numerical values. A visual element data structure may apply rules based on a comparison between such a ranking or numerical value and a threshold. For example, rankings may be applied to multiple potential conditions associated with high or low LVEF, and visual elements may be generated and/or ordered depending on such ranking.

Still referring to FIG. 1, in some embodiments, visual element may be interacted with. For example, visual element may include an interface, such as a button or menu. In some embodiments, visual element may be interacted with using a user device such as a smartphone.

Still referring to FIG. 1, in some embodiments, apparatus 100 may transmit visual element data structure to user device 188. In some embodiments, visual element data structure may configure user device 188 to display visual element. In some embodiments, visual element data structure may cause an event handler to be triggered in an application of user device 188 such as a web browser. In some embodiments, triggering of an event handler may cause a change in an application of user device 188 such as display of visual element.

Still referring to FIG. 1, in some embodiments, apparatus 100 may transmit visual element to a display. A display may communicate visual element to a user such as a doctor or subject 124. A display may include, for example, a smartphone screen, a computer screen, or a tablet screen. A display may be configured to provide a visual interface. A visual interface may include one or more virtual interactive elements such as, without limitation, buttons, menus, and the like. A display may include one or more physical interactive elements, such as buttons, a computer mouse, or a touchscreen, that allow a user to input data into the display. Interactive elements may be configured to enable interaction between a user and a computing device. In some embodiments, a visual element data structure is determined as a function of data input by a user into a display.

Still referring to FIG. 1, a variable and/or datum described herein may be represented as a data structure. In some embodiments, a data structure may include one or more functions and/or variables, as a class might in object-oriented programming. In some embodiments, a data structure may include data in the form of a Boolean, integer, float, string, date, and the like. In a non-limiting example, a LVEF prediction data structure may include a float value representing a numerical value of LVEF prediction 148. In some embodiments, data in a data structure may be organized in a linked list, tree, array, matrix, tenser, and the like. In some embodiments, a data structure may include or be associated with one or more elements of metadata. A data structure may include one or more self-referencing data elements, which processor 104 may use in interpreting the data structure. In a non-limiting example, a data structure may include “<date>” and “</date>,” tags, indicating that the content between the tags is a date.

Still referring to FIG. 1, a data structure may be stored in, for example, memory 108 or a database. Database may be implemented, without limitation, as a relational database, a key-value retrieval database such as a NOSQL database, or any other format or structure for use as a database that a person skilled in the art would recognize as suitable upon review of the entirety of this disclosure. Database may alternatively or additionally be implemented using a distributed data storage protocol and/or data structure, such as a distributed hash table or the like. Database may include a plurality of data entries and/or records as described above. Data entries in a database may be flagged with or linked to one or more additional elements of information, which may be reflected in data entry cells and/or in linked tables such as tables related by one or more indices in a relational database. Persons skilled in the art, upon reviewing the entirety of this disclosure, will be aware of various ways in which data entries in a database may store, retrieve, organize, and/or reflect data and/or records as used herein, as well as categories and/or populations of data consistently with this disclosure.

Still referring to FIG. 1, in some embodiments, a data structure may be read and/or manipulated by processor 104. In a non-limiting example, a visual element data structure may be read, and a visual element of the visual element data structure may be displayed to a user.

Still referring to FIG. 1, in some embodiments, a data structure may be calibrated. In some embodiments, a data structure may be trained using a machine learning algorithm. In a non-limiting example, a data structure may include an array of data representing the biases of connections of a neural network. In this example, the neural network may be trained on a set of training data, and a back propagation algorithm may be used to modify the data in the array. Machine learning models and neural networks are described further herein.

Still referring to FIG. 1, in some embodiments, apparatus 100 may be used to periodically monitor a subject's LVEF. Periodic monitoring may be implemented by periodically recording ECG data of subject 124 and using LVEF prediction model 152 to generate LVEF predictions based on such ECG data recordings. In some embodiments, ECG data of subject 124 may be captured every week, 2 weeks, 3 weeks, 4 weeks, 5 weeks, 6 weeks, 1 month, 2 months, 3 months, 4 months, 5 months, 6 months, 7 months, 8 months, 9 months, 10 months, 11 months, or 12 months. Such use of ECG data to periodically predict a subject's LVEF may make use of procedures for determining LVEF which are more intrusive, require more expertise, require rarer equipment, or the like unnecessary. In some embodiments, periodic monitoring may be performed on a subject on a treatment regimen including use of a drug which increases a risk of heart failure. In a non-limiting example, periodic monitoring may be performed on a subject on a treatment regimen including use of mavacamten. In some embodiments, periodic monitoring may be performed on a subject on a treatment regimen including use of a heart failure treatment drug. As used herein, a “heart failure treatment drug” is a drug used to treat heart failure, a drug used to prevent heart failure, or both. Non-limiting examples of heart failure treatment drugs include captopril, enalapril, fosinopril, lisinopril, perindopril, quinapril, ramipril, trandoapril, benazepril, moexipril, cadesartan, losartan, valsartan, sacubitril, ivabradine, acebutolol, atenolol, betaxolol, carvedilol, carvedilol phosphate, labetalol, metoprolol succinate, metoprolol tartrate, nadolol, nebivolol, pindolol, propranolol, empagliflozin, dapagliflozin, spironolactone, and eplerenone. In some embodiments, patient monitoring program may check the LVEF of a patient against a monitoring threshold value. For example, monitoring threshold value may be between 50 and 35%. In some embodiments, monitoring threshold value may be between 40% and 45%. In some embodiments, monitoring threshold value may be 45%. In some embodiments, monitoring threshold value may be 40%. In some embodiments, patient monitoring program may include monitoring a change in LVEF. In some embodiments, monitoring threshold value may include a change in LVEF. For example, monitoring threshold value may include a drop of 10% or more, a drop of 15% or more, a drop of 5% or more, a drop of 2% or more, and the like. In some embodiments, monitoring threshold value may be selected as a function of a patient's previous LVEF value. For example, if a patient's previous LVEF value was greater than 55%, then the monitoring threshold value may be selected to be a drop of 10% or more. For example, if a patient's previous LVEF value was between 45% and 55%, then the monitoring threshold value may be selected to be a drop of 5% or more. In some embodiments, monitoring threshold value may be selected as a function of a drug. In some embodiments, monitoring threshold value may be retrieved from a lookup table. For example, in some embodiments, lookup table may include drugs correlated to monitoring threshold values. In some embodiments, lookup table may include previous LVEF values correlated to monitoring threshold values.

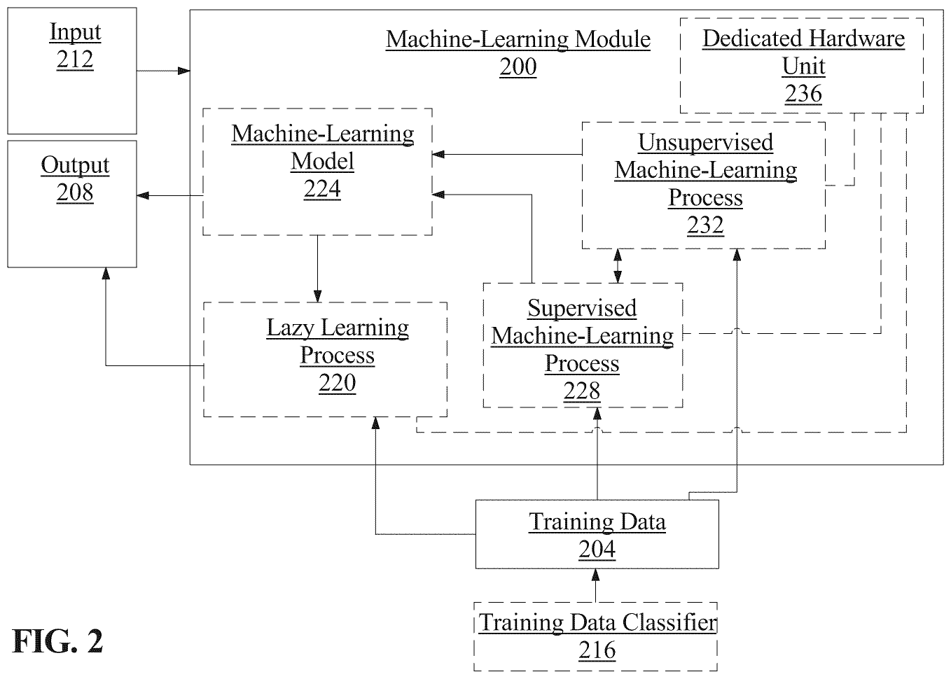

Referring now to FIG. 2, an exemplary embodiment of a machine-learning module 200 that may perform one or more machine-learning processes as described in this disclosure is illustrated. Machine-learning module may perform determinations, classification, and/or analysis steps, methods, processes, or the like as described in this disclosure using machine learning processes. A “machine learning process,” as used in this disclosure, is a process that automatedly uses training data 204 to generate an algorithm instantiated in hardware or software logic, data structures, and/or functions that will be performed by a computing device/module to produce outputs 208 given data provided as inputs 212; this is in contrast to a non-machine learning software program where the commands to be executed are determined in advance by a user and written in a programming language.

Still referring to FIG. 2, “training data,” as used herein, is data containing correlations that a machine-learning process may use to model relationships between two or more categories of data elements. For instance, and without limitation, training data 204 may include a plurality of data entries, also known as “training examples,” each entry representing a set of data elements that were recorded, received, and/or generated together; data elements may be correlated by shared existence in a given data entry, by proximity in a given data entry, or the like. Multiple data entries in training data 204 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 204 according to various correlations; correlations may indicate causative and/or predictive links between categories of data elements, which may be modeled as relationships such as mathematical relationships by machine-learning processes as described in further detail below. Training data 204 may be formatted and/or organized by categories of data elements, for instance by associating data elements with one or more descriptors corresponding to categories of data elements. As a non-limiting example, training data 204 may include data entered in standardized forms by persons or processes, such that entry of a given data element in a given field in a form may be mapped to one or more descriptors of categories. Elements in training data 204 may be linked to descriptors of categories by tags, tokens, or other data elements; for instance, and without limitation, training data 204 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively or additionally, and continuing to refer to FIG. 2, training data 204 may include one or more elements that are not categorized; that is, training data 204 may not be formatted or contain descriptors for some elements of data. Machine-learning algorithms and/or other processes may sort training data 204 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 204 to be made applicable for two or more distinct machine-learning algorithms as described in further detail below. Training data 204 used by machine-learning module 200 may correlate any input data as described in this disclosure to any output data as described in this disclosure. As a non-limiting illustrative example, an input may include an ECG datum and an output may include an LVEF prediction.

Further referring to FIG. 2, training data may be filtered, sorted, and/or selected using one or more supervised and/or unsupervised machine-learning processes and/or models as described in further detail below; such models may include without limitation a training data classifier 216. Training data classifier 216 may include a “classifier,” which as used in this disclosure is a machine-learning model as defined below, such as a data structure representing and/or using a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. A distance metric may include any norm, such as, without limitation, a Pythagorean norm. Machine-learning module 200 may generate a classifier using a classification algorithm, defined as a processes whereby a computing device and/or any module and/or component operating thereon derives a classifier from training data 204. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 216 may classify elements of training data to particular categories of subject such as based on demographics.

Still referring to FIG. 2, Computing device may be configured to generate a classifier using a Naïve Bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P (A/B)=P(B/A) P(A)÷P(B), where P (A/B) is the probability of hypothesis A given data B also known as posterior probability; P (B/A) is the probability of data B given that the hypothesis A was true; P (A) is the probability of hypothesis A being true regardless of data also known as prior probability of A; and P (B) is the probability of the data regardless of the hypothesis. A naïve Bayes algorithm may be generated by first transforming training data into a frequency table. Computing device may then calculate a likelihood table by calculating probabilities of different data entries and classification labels. Computing device may utilize a naïve Bayes equation to calculate a posterior probability for each class. A class containing the highest posterior probability is the outcome of prediction. Naïve Bayes classification algorithm may include a gaussian model that follows a normal distribution. Naïve Bayes classification algorithm may include a multinomial model that is used for discrete counts. Naïve Bayes classification algorithm may include a Bernoulli model that may be utilized when vectors are binary.

With continued reference to FIG. 2, Computing device may be configured to generate a classifier using a K-nearest neighbors (KNN) algorithm. A “K-nearest neighbors algorithm” as used in this disclosure, includes a classification method that utilizes feature similarity to analyze how closely out-of-sample-features resemble training data to classify input data to one or more clusters and/or categories of features as represented in training data; this may be performed by representing both training data and input data in vector forms, and using one or more measures of vector similarity to identify classifications within training data, and to determine a classification of input data. K-nearest neighbors algorithm may include specifying a K-value, or a number directing the classifier to select the k most similar entries training data to a given sample, determining the most common classifier of the entries in the database, and classifying the known sample; this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a non-limiting example, an initial heuristic may include a ranking of associations between inputs and elements of training data. Heuristic may include selecting some number of highest-ranking associations and/or training data elements.

With continued reference to FIG. 2, generating k-nearest neighbors algorithm may generate a first vector output containing a data entry cluster, generating a second vector output containing an input data, and calculate the distance between the first vector output and the second vector output using any suitable norm such as cosine similarity, Euclidean distance measurement, or the like. Each vector output may be represented, without limitation, as an n-tuple of values, where n is at least two values. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute l as derived using a Pythagorean norm:

$$l = \sqrt{{\sum }_{i = 0}^{n} {a}_{i}^{2}} ,$$

where aᵢ is attribute number i of the vector. Scaling and/or normalization may function to make vector comparison independent of absolute quantities of attributes, while preserving any dependency on similarity of attributes; this may, for instance, be advantageous where cases represented in training data are represented by different quantities of samples, which may result in proportionally equivalent vectors with divergent values.

With further reference to FIG. 2, training examples for use as training data may be selected from a population of potential examples according to cohorts relevant to an analytical problem to be solved, a classification task, or the like. Alternatively or additionally, training data may be selected to span a set of likely circumstances or inputs for a machine-learning model and/or process to encounter when deployed. For instance, and without limitation, for each category of input data to a machine-learning process or model that may exist in a range of values in a population of phenomena such as images, user data, process data, physical data, or the like, a computing device, processor, and/or machine-learning model may select training examples representing each possible value on such a range and/or a representative sample of values on such a range. Selection of a representative sample may include selection of training examples in proportions matching a statistically determined and/or predicted distribution of such values according to relative frequency, such that, for instance, values encountered more frequently in a population of data so analyzed are represented by more training examples than values that are encountered less frequently. Alternatively or additionally, a set of training examples may be compared to a collection of representative values in a database and/or presented to a user, so that a process can detect, automatically or via user input, one or more values that are not included in the set of training examples. Computing device, processor, and/or module may automatically generate a missing training example; this may be done by receiving and/or retrieving a missing input and/or output value and correlating the missing input and/or output value with a corresponding output and/or input value collocated in a data record with the retrieved value, provided by a user and/or other device, or the like.

Continuing to refer to FIG. 2, computer, processor, and/or module may be configured to preprocess training data. “Preprocessing” training data, as used in this disclosure, is transforming training data from raw form to a format that can be used for training a machine learning model. Preprocessing may include sanitizing, feature selection, feature scaling, data augmentation and the like.

Still referring to FIG. 2, computer, processor, and/or module may be configured to sanitize training data. “Sanitizing” training data, as used in this disclosure, is a process whereby training examples are removed that interfere with convergence of a machine-learning model and/or process to a useful result. For instance, and without limitation, a training example may include an input and/or output value that is an outlier from typically encountered values, such that a machine-learning algorithm using the training example will be adapted to an unlikely amount as an input and/or output; a value that is more than a threshold number of standard deviations away from an average, mean, or expected value, for instance, may be eliminated. Alternatively or additionally, one or more training examples may be identified as having poor quality data, where “poor quality” is defined as having a signal to noise ratio below a threshold value. Sanitizing may include steps such as removing duplicative or otherwise redundant data, interpolating missing data, correcting data errors, standardizing data, identifying outliers, and the like. In a nonlimiting example, sanitization may include utilizing algorithms for identifying duplicate entries or spell-check algorithms.

As a non-limiting example, and with further reference to FIG. 2, images used to train an image classifier or other machine-learning model and/or process that takes images as inputs or generates images as outputs may be rejected if image quality is below a threshold value. For instance, and without limitation, computing device, processor, and/or module may perform blur detection, and eliminate one or more Blur detection may be performed, as a non-limiting example, by taking Fourier transform, or an approximation such as a Fast Fourier Transform (FFT) of the image and analyzing a distribution of low and high frequencies in the resulting frequency-domain depiction of the image; numbers of high-frequency values below a threshold level may indicate blurriness. As a further non-limiting example, detection of blurriness may be performed by convolving an image, a channel of an image, or the like with a Laplacian kernel; this may generate a numerical score reflecting a number of rapid changes in intensity shown in the image, such that a high score indicates clarity and a low score indicates blurriness. Blurriness detection may be performed using a gradient-based operator, which measures operators based on the gradient or first derivative of an image, based on the hypothesis that rapid changes indicate sharp edges in the image, and thus are indicative of a lower degree of blurriness. Blur detection may be performed using Wavelet-based operator, which takes advantage of the capability of coefficients of the discrete wavelet transform to describe the frequency and spatial content of images. Blur detection may be performed using statistics-based operators take advantage of several image statistics as texture descriptors in order to compute a focus level. Blur detection may be performed by using discrete cosine transform (DCT) coefficients in order to compute a focus level of an image from its frequency content.

Continuing to refer to FIG. 2, computing device, processor, and/or module may be configured to precondition one or more training examples. For instance, and without limitation, where a machine learning model and/or process has one or more inputs and/or outputs requiring, transmitting, or receiving a certain number of bits, samples, or other units of data, one or more training examples' elements to be used as or compared to inputs and/or outputs may be modified to have such a number of units of data. For instance, a computing device, processor, and/or module may convert a smaller number of units, such as in a low pixel count image, into a desired number of units, for instance by upsampling and interpolating. As a non-limiting example, a low pixel count image may have 100 pixels, however a desired number of pixels may be 128. Processor may interpolate the low pixel count image to convert the 100 pixels into 128 pixels. It should also be noted that one of ordinary skill in the art, upon reading this disclosure, would know the various methods to interpolate a smaller number of data units such as samples, pixels, bits, or the like to a desired number of such units. In some instances, a set of interpolation rules may be trained by sets of highly detailed inputs and/or outputs and corresponding inputs and/or outputs downsampled to smaller numbers of units, and a neural network or other machine learning model that is trained to predict interpolated pixel values using the training data. As a non-limiting example, a sample input and/or output, such as a sample picture, with sample-expanded data units (e.g., pixels added between the original pixels) may be input to a neural network or machine-learning model and output a pseudo replica sample-picture with dummy values assigned to pixels between the original pixels based on a set of interpolation rules. As a non-limiting example, in the context of an image classifier, a machine-learning model may have a set of interpolation rules trained by sets of highly detailed images and images that have been downsampled to smaller numbers of pixels, and a neural network or other machine learning model that is trained using those examples to predict interpolated pixel values in a facial picture context. As a result, an input with sample-expanded data units (the ones added between the original data units, with dummy values) may be run through a trained neural network and/or model, which may fill in values to replace the dummy values. Alternatively or additionally, processor, computing device, and/or module may utilize sample expander methods, a low-pass filter, or both. As used in this disclosure, a “low-pass filter” is a filter that passes signals with a frequency lower than a selected cutoff frequency and attenuates signals with frequencies higher than the cutoff frequency. The exact frequency response of the filter depends on the filter design. Computing device, processor, and/or module may use averaging, such as luma or chroma averaging in images, to fill in data units in between original data units.

In some embodiments, and with continued reference to FIG. 2, computing device, processor, and/or module may down-sample elements of a training example to a desired lower number of data elements. As a non-limiting example, a high pixel count image may have 256 pixels, however a desired number of pixels may be 128. Processor may down-sample the high pixel count image to convert the 256 pixels into 128 pixels. In some embodiments, processor may be configured to perform downsampling on data. Downsampling, also known as decimation, may include removing every Nth entry in a sequence of samples, all but every Nth entry, or the like, which is a process known as “compression,” and may be performed, for instance by an N-sample compressor implemented using hardware or software. Anti-aliasing and/or anti-imaging filters, and/or low-pass filters, may be used to clean up side-effects of compression.

Further referring to FIG. 2, feature selection includes narrowing and/or filtering training data to exclude features and/or elements, or training data including such elements, that are not relevant to a purpose for which a trained machine-learning model and/or algorithm is being trained, and/or collection of features and/or elements, or training data including such elements, on the basis of relevance or utility for an intended task or purpose for a trained machine-learning model and/or algorithm is being trained. Feature selection may be implemented, without limitation, using any process described in this disclosure, including without limitation using training data classifiers, exclusion of outliers, or the like.

With continued reference to FIG. 2, feature scaling may include, without limitation, normalization of data entries, which may be accomplished by dividing numerical fields by norms thereof, for instance as performed for vector normalization. Feature scaling may include absolute maximum scaling, wherein each quantitative datum is divided by the maximum absolute value of all quantitative data of a set or subset of quantitative data. Feature scaling may include min-max scaling, in which each value X has a minimum value Xₘᵢₙ in a set or subset of values subtracted therefrom, with the result divided by the range of the values, give maximum value in the set or subset

$${X}_{max} : {X}_{new} = \frac{X − {X}_{min}}{{X}_{max} − {X}_{min}} .$$

Feature scaling may include mean normalization, which involves use of a mean value of a set and/or subset of values, Xₘₑₐₙ with maximum and minimum values:

$${X}_{new} = \frac{X − {X}_{mean}}{{X}_{max} − {X}_{min}} .$$

Feature scaling may include standardization, where a difference between X and Xₘₑₐₙ is divided by a standard deviation σ of a set or subset of values:

$${X}_{new} = \frac{X − {X}_{mean}}{σ} .$$

Scaling may be performed using a median value of a set or subset X_{median} and/or interquartile range (IQR), which represents the difference between the 25^{th} percentile value and the 50^{th} percentile value (or closest values thereto by a rounding protocol), such as:

$${X}_{new} = \frac{X − {X}_{median}}{IQR} .$$

Persons skilled in the art, upon reviewing the entirety of this disclosure, will be aware of various alternative or additional approaches that may be used for feature scaling.

Further referring to FIG. 2, computing device, processor, and/or module may be configured to perform one or more processes of data augmentation. “Data augmentation” as used in this disclosure is addition of data to a training set using elements and/or entries already in the dataset. Data augmentation may be accomplished, without limitation, using interpolation, generation of modified copies of existing entries and/or examples, and/or one or more generative AI processes, for instance using deep neural networks and/or generative adversarial networks; generative processes may be referred to alternatively in this context as “data synthesis” and as creating “synthetic data.” Augmentation may include performing one or more transformations on data, such as geometric, color space, affine, brightness, cropping, and/or contrast transformations of images.

Still referring to FIG. 2, machine-learning module 200 may be configured to perform a lazy-learning process 220 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 204. Heuristic may include selecting some number of highest-ranking associations and/or training data 204 elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively or additionally, and with continued reference to FIG. 2, machine-learning processes as described in this disclosure may be used to generate machine-learning models 224. A “machine-learning model,” as used in this disclosure, is a data structure representing and/or instantiating a mathematical and/or algorithmic representation of a relationship between inputs and outputs, as generated using any machine-learning process including without limitation any process as described above, and stored in memory; an input is submitted to a machine-learning model 224 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 224 may be generated by creating an artificial neural network, such as a convolutional neural network comprising an input layer of nodes, one or more intermediate layers, and an output layer of nodes. Connections between nodes may be created via the process of “training” the network, in which elements from a training data 204 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 2, machine-learning algorithms may include at least a supervised machine-learning process 228. At least a supervised machine-learning process 228, as defined herein, include algorithms that receive a training set relating a number of inputs to a number of outputs, and seek to generate one or more data structures representing and/or instantiating one or more mathematical relations relating inputs to outputs, where each of the one or more mathematical relations is optimal according to some criterion specified to the algorithm using some scoring function. For instance, a supervised learning algorithm may include ECG data as described above as inputs, LVEF predictions as outputs, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 204. Persons skilled in the art, upon reviewing the entirety of this disclosure, will be aware of various possible variations of at least a supervised machine-learning process 228 that may be used to determine relation between inputs and outputs. Supervised machine-learning processes may include classification algorithms as defined above.

With further reference to FIG. 2, training a supervised machine-learning process may include, without limitation, iteratively updating coefficients, biases, weights based on an error function, expected loss, and/or risk function. For instance, an output generated by a supervised machine-learning model using an input example in a training example may be compared to an output example from the training example; an error function may be generated based on the comparison, which may include any error function suitable for use with any machine-learning algorithm described in this disclosure, including a square of a difference between one or more sets of compared values or the like. Such an error function may be used in turn to update one or more weights, biases, coefficients, or other parameters of a machine-learning model through any suitable process including without limitation gradient descent processes, least-squares processes, and/or other processes described in this disclosure. This may be done iteratively and/or recursively to gradually tune such weights, biases, coefficients, or other parameters. Updating may be performed, in neural networks, using one or more back-propagation algorithms. Iterative and/or recursive updates to weights, biases, coefficients, or other parameters as described above may be performed until currently available training data is exhausted and/or until a convergence test is passed, where a “convergence test” is a test for a condition selected as indicating that a model and/or weights, biases, coefficients, or other parameters thereof has reached a degree of accuracy. A convergence test may, for instance, compare a difference between two or more successive errors or error function values, where differences below a threshold amount may be taken to indicate convergence. Alternatively or additionally, one or more errors and/or error function values evaluated in training iterations may be compared to a threshold.

Still referring to FIG. 2, a computing device, processor, and/or module may be configured to perform method, method step, sequence of method steps and/or algorithm described in reference to this figure, in any order and with any degree of repetition. For instance, a computing device, processor, and/or module may be configured to perform a single step, sequence and/or algorithm repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. A computing device, processor, and/or module may perform any step, sequence of steps, or algorithm in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Further referring to FIG. 2, machine learning processes may include at least an unsupervised machine-learning processes 232. An unsupervised machine-learning process, as used herein, is a process that derives inferences in datasets without regard to labels; as a result, an unsupervised machine-learning process may be free to discover any structure, relationship, and/or correlation provided in the data. Unsupervised processes 232 may not require a response variable; unsupervised processes 232 may be used to find interesting patterns and/or inferences between variables, to determine a degree of correlation between two or more variables, or the like.

Still referring to FIG. 2, machine-learning module 200 may be designed and configured to create a machine-learning model 224 using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 2, machine-learning algorithms may include, without limitation, linear discriminant analysis. Machine-learning algorithm may include quadratic discriminant analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include various forms of latent space regularization such as variational regularization. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized trees, AdaBoost, gradient tree boosting, and/or voting classifier methods. Machine-learning algorithms may include neural net algorithms, including convolutional neural net processes.

Still referring to FIG. 2, a machine-learning model and/or process may be deployed or instantiated by incorporation into a program, apparatus, system and/or module. For instance, and without limitation, a machine-learning model, neural network, and/or some or all parameters thereof may be stored and/or deployed in any memory or circuitry. Parameters such as coefficients, weights, and/or biases may be stored as circuit-based constants, such as arrays of wires and/or binary inputs and/or outputs set at logic “1” and “0” voltage levels in a logic circuit to represent a number according to any suitable encoding system including twos complement or the like or may be stored in any volatile and/or non-volatile memory. Similarly, mathematical operations and input and/or output of data to or from models, neural network layers, or the like may be instantiated in hardware circuitry and/or in the form of instructions in firmware, machine-code such as binary operation code instructions, assembly language, or any higher-order programming language. Any technology for hardware and/or software instantiation of memory, instructions, data structures, and/or algorithms may be used to instantiate a machine-learning process and/or model, including without limitation any combination of production and/or configuration of non-reconfigurable hardware elements, circuits, and/or modules such as without limitation ASICs, production and/or configuration of reconfigurable hardware elements, circuits, and/or modules such as without limitation FPGAs, production and/or of non-reconfigurable and/or configuration non-rewritable memory elements, circuits, and/or modules such as without limitation non-rewritable ROM, production and/or configuration of reconfigurable and/or rewritable memory elements, circuits, and/or modules such as without limitation rewritable ROM or other memory technology described in this disclosure, and/or production and/or configuration of any computing device and/or component thereof as described in this disclosure. Such deployed and/or instantiated machine-learning model and/or algorithm may receive inputs from any other process, module, and/or component described in this disclosure, and produce outputs to any other process, module, and/or component described in this disclosure.

Continuing to refer to FIG. 2, any process of training, retraining, deployment, and/or instantiation of any machine-learning model and/or algorithm may be performed and/or repeated after an initial deployment and/or instantiation to correct, refine, and/or improve the machine-learning model and/or algorithm. Such retraining, deployment, and/or instantiation may be performed as a periodic or regular process, such as retraining, deployment, and/or instantiation at regular elapsed time periods, after some measure of volume such as a number of bytes or other measures of data processed, a number of uses or performances of processes described in this disclosure, or the like, and/or according to a software, firmware, or other update schedule. Alternatively or additionally, retraining, deployment, and/or instantiation may be event-based, and may be triggered, without limitation, by user inputs indicating sub-optimal or otherwise problematic performance and/or by automated field testing and/or auditing processes, which may compare outputs of machine-learning models and/or algorithms, and/or errors and/or error functions thereof, to any thresholds, convergence tests, or the like, and/or may compare outputs of processes described herein to similar thresholds, convergence tests or the like. Event-based retraining, deployment, and/or instantiation may alternatively or additionally be triggered by receipt and/or generation of one or more new training examples; a number of new training examples may be compared to a preconfigured threshold, where exceeding the preconfigured threshold may trigger retraining, deployment, and/or instantiation.

Still referring to FIG. 2, retraining and/or additional training may be performed using any process for training described above, using any currently or previously deployed version of a machine-learning model and/or algorithm as a starting point. Training data for retraining may be collected, preconditioned, sorted, classified, sanitized or otherwise processed according to any process described in this disclosure. Training data may include, without limitation, training examples including inputs and correlated outputs used, received, and/or generated from any version of any system, module, machine-learning model or algorithm, apparatus, and/or method described in this disclosure; such examples may be modified and/or labeled according to user feedback or other processes to indicate desired results, and/or may have actual or measured results from a process being modeled and/or predicted by system, module, machine-learning model or algorithm, apparatus, and/or method as “desired” results to be compared to outputs for training processes as described above.

Redeployment may be performed using any reconfiguring and/or rewriting of reconfigurable and/or rewritable circuit and/or memory elements; alternatively, redeployment may be performed by production of new hardware and/or software components, circuits, instructions, or the like, which may be added to and/or may replace existing hardware and/or software components, circuits, instructions, or the like.

Further referring to FIG. 2, one or more processes or algorithms described above may be performed by at least a dedicated hardware unit 236. A “dedicated hardware unit,” for the purposes of this figure, is a hardware component, circuit, or the like, aside from a principal control circuit and/or processor performing method steps as described in this disclosure, that is specifically designated or selected to perform one or more specific tasks and/or processes described in reference to this figure, such as without limitation preconditioning and/or sanitization of training data and/or training a machine-learning algorithm and/or model. A dedicated hardware unit 236 may include, without limitation, a hardware unit that can perform iterative or massed calculations, such as matrix-based calculations to update or tune parameters, weights, coefficients, and/or biases of machine-learning models and/or neural networks, efficiently using pipelining, parallel processing, or the like; such a hardware unit may be optimized for such processes by, for instance, including dedicated circuitry for matrix and/or signal processing operations that includes, e.g., multiple arithmetic and/or logical circuit units such as multipliers and/or adders that can act simultaneously and/or in parallel or the like. Such dedicated hardware units 236 may include, without limitation, graphical processing units (GPUs), dedicated signal processing modules, FPGA or other reconfigurable hardware that has been configured to instantiate parallel processing units for one or more specific tasks, or the like, A computing device, processor, apparatus, or module may be configured to instruct one or more dedicated hardware units 236 to perform one or more operations described herein, such as evaluation of model and/or algorithm outputs, one-time or iterative updates to parameters, coefficients, weights, and/or biases, and/or any other operations such as vector and/or matrix operations as described in this disclosure.

With continued reference to FIG. 2, apparatus 100 may use user feedback to train the machine-learning models and/or classifiers described above. For example, classifier may be trained using past inputs and outputs of classifier. In some embodiments, if user feedback indicates that an output of classifier was “bad,” then that output and the corresponding input may be removed from training data used to train classifier, and/or may be replaced with a value entered by, e.g., another user that represents an ideal output given the input the classifier originally received, permitting use in retraining, and adding to training data; in either case, classifier may be retrained with modified training data as described in further detail below. In some embodiments, training data of classifier may include user feedback.

With continued reference to FIG. 2, in some embodiments, an accuracy score may be calculated for classifier using user feedback. For the purposes of this disclosure, “accuracy score,” is a numerical value concerning the accuracy of a machine-learning model. For example, a plurality of user feedback scores may be averaged to determine an accuracy score. In some embodiments, a cohort accuracy score may be determined for particular cohorts of persons. For example, user feedback for users belonging to a particular cohort of persons may be averaged together to determine the cohort accuracy score for that particular cohort of persons and used as described above. Accuracy score or another score as described above may indicate a degree of retraining needed for a machine-learning model such as a classifier; apparatus 100 may perform a larger number of retraining cycles for a higher number (or lower number, depending on a numerical interpretation used), and/or may collect more training data for such retraining, perform more training cycles, apply a more stringent convergence test such as a test requiring a lower mean squared error, and/or indicate to a user and/or operator that additional training data is needed.

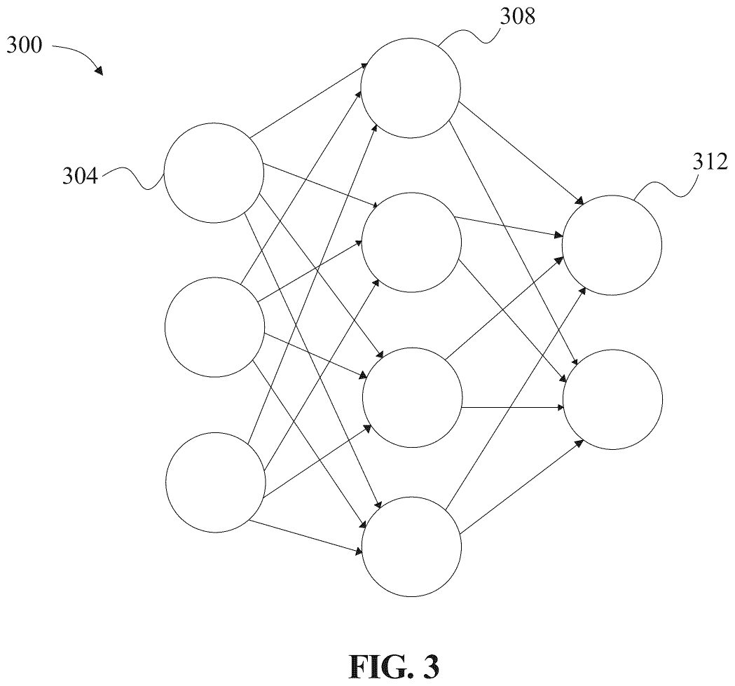

Referring now to FIG. 3, an exemplary embodiment of neural network 300 is illustrated. A neural network 300 also known as an artificial neural network, is a network of “nodes,” or data structures having one or more inputs, one or more outputs, and a function determining outputs based on inputs. Such nodes may be organized in a network, such as without limitation a convolutional neural network, including an input layer of nodes 304, one or more intermediate layers 308, and an output layer of nodes 312. Connections between nodes may be created via the process of “training” the network, in which elements from a training dataset are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning. Connections may run solely from input nodes toward output nodes in a “feed-forward” network or may feed outputs of one layer back to inputs of the same or a different layer in a “recurrent network.” As a further non-limiting example, a neural network may include a convolutional neural network comprising an input layer of nodes, one or more intermediate layers, and an output layer of nodes. A “convolutional neural network,” as used in this disclosure, is a neural network in which at least one hidden layer is a convolutional layer that convolves inputs to that layer with a subset of inputs known as a “kernel,” along with one or more additional layers such as pooling layers, fully connected layers, and the like.

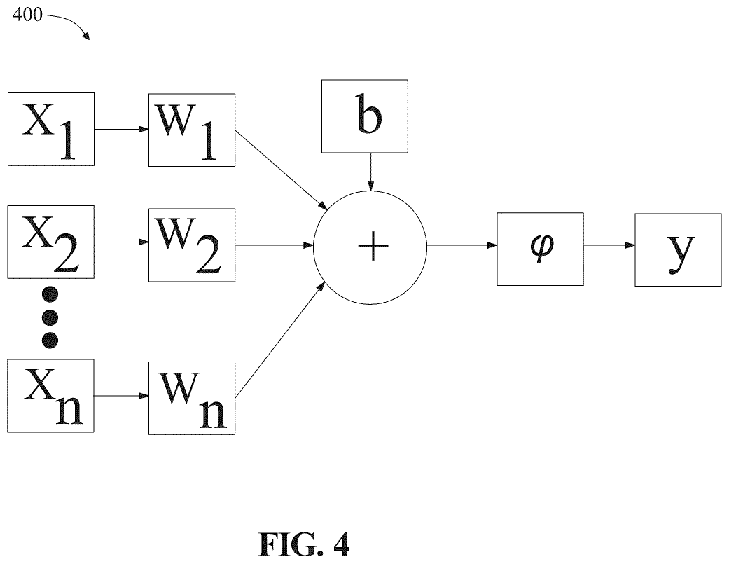

Referring now to FIG. 4, an exemplary embodiment of a node 400 of a neural network is illustrated. A node may include, without limitation a plurality of inputs xᵢ that may receive numerical values from inputs to a neural network containing the node and/or from other nodes. Node may perform one or more activation functions to produce its output given one or more inputs, such as without limitation computing a binary step function comparing an input to a threshold value and outputting either a logic 1 or logic 0 output or something equivalent, a linear activation function whereby an output is directly proportional to the input, and/or a non-linear activation function, wherein the output is not proportional to the input. Non-linear activation functions may include, without limitation, a sigmoid function of the form

$$f ( x ) = \frac{1}{1 - {e}^{- x}}$$

given input x, a tanh (hyperbolic tangent) function, of the form

$$\frac{{e}^{x} - {e}^{- x}}{{e}^{x} + {e}^{- x}} ,$$

a tanh derivative function such as ƒ(x)=tanh²(x), a rectified linear unit function such as ƒ(x)=max(0, x), a “leaky” and/or “parametric” rectified linear unit function such as ƒ(x)=max(ax, x) for some a, an exponential linear units function such as

$$f ( x ) = \{ \begin{matrix}x for x \geq 0 \\ α ( {e}^{x} - 1 ) for x < 0\end{matrix}$$

for some value of α (this function may be replaced and/or weighted by its own derivative in some embodiments), a softmax function such as

$$f ( {x}_{i} ) = \frac{{e}^{x}}{{\sum }_{i} {x}_{i}}$$

where the inputs to an instant layer are xᵢ, a swish function such as ƒ(x)=x*sigmoid(x), a Gaussian error linear unit function such as f(x)=a(1+tanh(\sqrt{2/π}(x+bx^{r}))) for some values of a, b, and r, and/or a scaled exponential linear unit function such as

$$f ( x ) = λ \{ \begin{matrix}α ( {e}^{x} - 1 ) for x < 0 \\ x for x \geq 0\end{matrix} .$$

Fundamentally, there is no limit to the nature of functions of inputs x; that may be used as activation functions. As a non-limiting and illustrative example, node may perform a weighted sum of inputs using weights wᵢ that are multiplied by respective inputs xᵢ. Additionally or alternatively, a bias b may be added to the weighted sum of the inputs such that an offset is added to each unit in the neural network layer that is independent of the input to the layer. The weighted sum may then be input into a function φ, which may generate one or more outputs y. Weight wᵢ applied to an input xᵢ may indicate whether the input is “excitatory,” indicating that it has strong influence on the one or more outputs y, for instance by the corresponding weight having a large numerical value, and/or a “inhibitory,” indicating it has a weak effect influence on the one more inputs y, for instance by the corresponding weight having a small numerical value. The values of weights wᵢ may be determined by training a neural network using training data, which may be performed using any suitable process as described above.

Still referring to FIG. 4, a “convolutional neural network,” as used in this disclosure, is a neural network in which at least one hidden layer is a convolutional layer that convolves inputs to that layer with a subset of inputs known as a “kernel,” along with one or more additional layers such as pooling layers, fully connected layers, and the like. CNN may include, without limitation, a deep neural network (DNN) extension, where a DNN is defined as a neural network with two or more hidden layers.

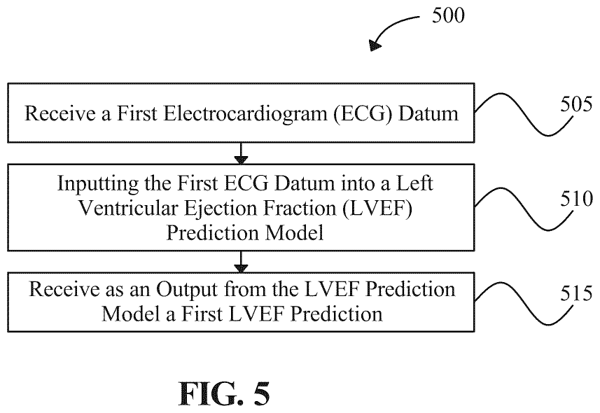

Referring now to FIG. 5, an exemplary embodiment of a method 500 of left ventricular ejection fraction (LVEF) prediction is illustrated. One or more steps if method 500 may be implemented, without limitation, as described with reference to other figures. One or more steps of method 500 may be implemented, without limitation, using at least a processor.

Still referring to FIG. 5, in some embodiments, method 500 may include a step 505 of receiving a first electrocardiogram (ECG) datum.

Still referring to FIG. 5, in some embodiments, method 500 may include a step 510 of inputting the first ECG datum into an LVEF prediction model.

Still referring to FIG. 5, in some embodiments, method 500 may include a step 515 of receiving as an output from the LVEF prediction model a first LVEF prediction, wherein the first LVEF prediction comprises a confidence metric.

Systems and methods of this disclosure may be consistent with any systems or methods disclosed in U.S. patent application Ser. No. 18/665,932 filed on May 16, 2024, and titled “SYSTEMS AND METHODS FOR TRAINING MACHINE LEARNING MODELS USING UNLABELED ELECTROCARDIOGRAM DATA”, the entirety of which is hereby incorporated by reference.

Still referring to FIG. 5, in some embodiments, method 500 further includes determining a diastolic dysfunction datum if the first LVEF prediction is above 50%. In some embodiments, method 500 further includes determining a left ventricular dysfunction datum if the first LVEF prediction is below 50%. In some embodiments, method 500 further includes determining a cardiac assessment datum if the first LVEF prediction is below 45%. In some embodiments, method 500 further includes determining a left ventricular dysfunction datum if the first LVEF prediction is below 40%. In some embodiments, method 500 further includes determining a sudden death risk datum if the first LVEF prediction is below 35%. In some embodiments, method 500 further includes determining a left ventricular dysfunction datum if the first LVEF prediction is below 30%. In some embodiments, method 500 further includes using the at least a processor, receiving a second electrocardiogram (ECG) datum; using the at least a processor, inputting the second ECG datum into an LVEF prediction model; using the at least a processor, receiving as an output from the LVEF prediction model a second LVEF prediction; and using the at least a processor, determining a LVEF decline datum if the second LVEF prediction is at least 5% lower than the first LVEF prediction; wherein the second ECG datum is recorded after the first ECG datum; wherein the first LVEF prediction is determined as a function of the first ECG datum; and wherein the second LVEF prediction is determined as a function of the second ECG datum. In some embodiments, method 500 further includes using the at least a processor, receiving a second electrocardiogram (ECG) datum; using the at least a processor, inputting the second ECG datum into an LVEF prediction model; using the at least a processor, receiving as an output from the LVEF prediction model a second LVEF prediction; and using the at least a processor, determining a LVEF decline datum if the second LVEF prediction is at least 10% lower than the first LVEF prediction; wherein the second ECG datum is recorded after the first ECG datum; wherein the first LVEF prediction is determined as a function of the first ECG datum; and wherein the second LVEF prediction is determined as a function of the second ECG datum. In some embodiments, method 500 further includes using the at least a processor, training the LVEF prediction model on a training dataset including a plurality of example ECG data as inputs correlated to a plurality of example LVEF levels as outputs; and using the at least a processor, generating the LVEF prediction as a function of the ECG datum using the trained LVEF prediction model. In some embodiments, method 500 further includes periodically monitoring a subject's LVEF using the LVEF prediction model. In some embodiments, the subject is on a treatment regimen including use of a drug which increases a risk of heart failure. In some embodiments, the subject is on a treatment regimen including use of a heart failure treatment drug.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

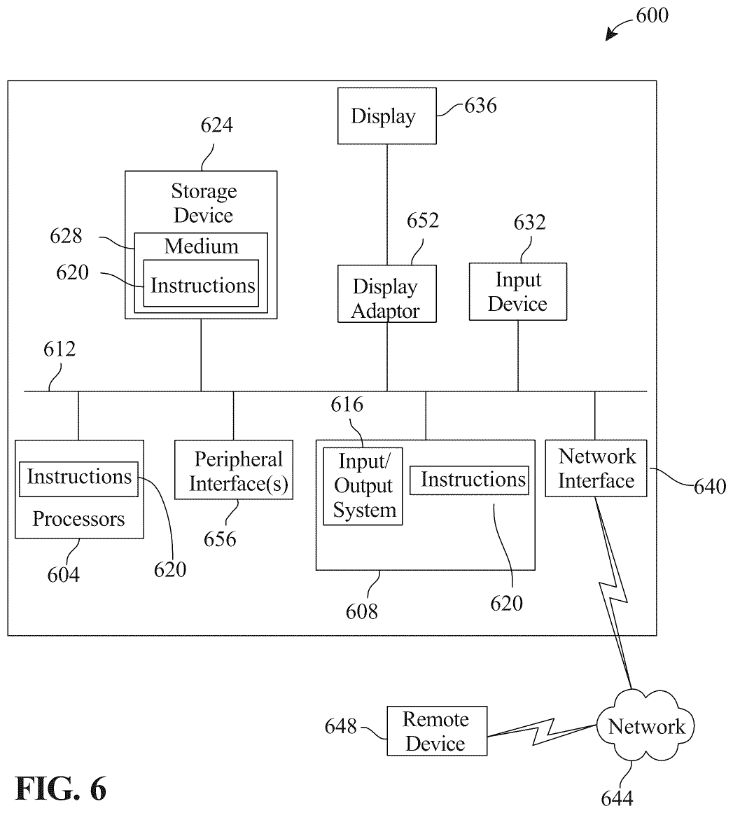

FIG. 6 shows a diagrammatic representation of one embodiment of a computing device in the exemplary form of a computer system 600 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 600 includes a processor 604 and a memory 608 that communicate with each other, and with other components, via a bus 612. Bus 612 may include any of several types of bus structures including, but not limited to, a memory bus, a memory controller, a peripheral bus, a local bus, and any combinations thereof, using any of a variety of bus architectures.

Processor 604 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 604 may be organized according to Von Neumann and/or Harvard architecture as a non-limiting example. Processor 604 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), and/or system on a chip (SoC).

Memory 608 may include various components (e.g., machine-readable media) including, but not limited to, a random-access memory component, a read only component, and any combinations thereof. In one example, a basic input/output system 616 (BIOS), including basic routines that help to transfer information between elements within computer system 600, such as during start-up, may be stored in memory 608. Memory 608 may also include (e.g., stored on one or more machine-readable media) instructions (e.g., software) 620 embodying any one or more of the aspects and/or methodologies of the present disclosure. In another example, memory 608 may further include any number of program modules including, but not limited to, an operating system, one or more application programs, other program modules, program data, and any combinations thereof.

Computer system 600 may also include a storage device 624. Examples of a storage device (e.g., storage device 624) include, but are not limited to, a hard disk drive, a magnetic disk drive, an optical disc drive in combination with an optical medium, a solid-state memory device, and any combinations thereof. Storage device 624 may be connected to bus 612 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 624 (or one or more components thereof) may be removably interfaced with computer system 600 (e.g., via an external port connector (not shown)). Particularly, storage device 624 and an associated machine-readable medium 628 may provide nonvolatile and/or volatile storage of machine-readable instructions, data structures, program modules, and/or other data for computer system 600. In one example, software 620 may reside, completely or partially, within machine-readable medium 628. In another example, software 620 may reside, completely or partially, within processor 604.

Computer system 600 may also include an input device 632. In one example, a user of computer system 600 may enter commands and/or other information into computer system 600 via input device 632. Examples of an input device 632 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 632 may be interfaced to bus 612 via any of a variety of interfaces (not shown) including, but not limited to, a serial interface, a parallel interface, a game port, a USB interface, a FIREWIRE interface, a direct interface to bus 612, and any combinations thereof. Input device 632 may include a touch screen interface that may be a part of or separate from display device 636, discussed further below. Input device 632 may be utilized as a user selection device for selecting one or more graphical representations in a graphical interface as described above.

A user may also input commands and/or other information to computer system 600 via storage device 624 (e.g., a removable disk drive, a flash drive, etc.) and/or network interface device 640. A network interface device, such as network interface device 640, may be utilized for connecting computer system 600 to one or more of a variety of networks, such as network 644, and one or more remote devices 648 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 644, may employ a wired and/or a wireless mode of communication. In general, any network topology may be used. Information (e.g., data, software 620, etc.) may be communicated to and/or from computer system 600 via network interface device 640.

Computer system 600 may further include a video display adapter 652 for communicating a displayable image to a display device, such as display device 636. Examples of a display device include, but are not limited to, a liquid crystal display (LCD), a cathode ray tube (CRT), a plasma display, a light emitting diode (LED) display, and any combinations thereof. Display adapter 652 and display device 636 may be utilized in combination with processor 604 to provide graphical representations of aspects of the present disclosure. In addition to a display device, computer system 600 may include one or more other peripheral output devices including, but not limited to, an audio speaker, a printer, and any combinations thereof. Such peripheral output devices may be connected to bus 612 via a peripheral interface 656. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

1. An apparatus for left ventricular ejection fraction (LVEF) prediction, the apparatus comprising:
at least one processor; and
a memory communicatively connected to the at least one processor, wherein the memory contains instructions configuring the at least one processor to:
receive a first electrocardiogram (ECG) datum;
input the first ECG datum into a LVEF prediction model comprising a masked autoencoder model, the masked autoencoder model configured to receive the first ECG datum in a modified form having masked temporal patches, reconstruct the first ECG datum including the masked temporal patches, and remove noise from the ECG datum by identifying reconstruction errors and adjusting for discrepancies, wherein the masked autoencoder model iteratively adjusts parameter values to minimize differences between reconstructed temporal patches and the masked temporal patches; and
generate a first LVEF prediction comprising a confidence metric using the LVEF prediction model.

2. The apparatus of claim 1, wherein the memory contains instructions configuring the at least one processor to determine a diastolic dysfunction datum if the first LVEF prediction is above 50%.

3. The apparatus of claim 1, wherein the memory contains instructions configuring the at least one processor to determine a left ventricular dysfunction datum if the first LVEF prediction is below 50%.

4. The apparatus of claim 1, wherein the memory contains instructions configuring the at least one processor to determine a cardiac assessment datum if the first LVEF prediction is below 45%.

5. The apparatus of claim 1, wherein the memory contains instructions configuring the at least one processor to determine a left ventricular dysfunction datum if the first LVEF prediction is below 40%.

6. The apparatus of claim 1, wherein the memory contains instructions configuring the at least one processor to determine a sudden death risk datum if the first LVEF prediction is below 35%.

7. The apparatus of claim 1, wherein the memory contains instructions configuring the at least one processor to determine a left ventricular dysfunction datum if the first LVEF prediction is below 30%.

8. The apparatus of claim 1, wherein the memory contains instructions configuring the at least one processor to:
receive a second electrocardiogram (ECG) datum recorded after the first ECG datum;
input the second ECG datum into the LVEF prediction model;
receive as an output from the LVEF prediction model a second LVEF prediction; and
determine a LVEF decline datum if the second LVEF prediction is at least 5% lower than the first LVEF prediction.

9. The apparatus of claim 1, wherein the memory contains instructions configuring the at least one processor to:
receive a second electrocardiogram (ECG) datum recorded after the first ECG datum;
input the second ECG datum into the LVEF prediction model;
receive as an output from the LVEF prediction model a second LVEF prediction; and
determine a LVEF decline datum if the second LVEF prediction is at least 10% lower than the first LVEF prediction, wherein the 10% decline threshold corresponds to a monitoring threshold value associated with patient drug regimens including mavacamten and heart failure treatment drugs.

10. (canceled)

11. The apparatus of claim 1, wherein the memory contains instructions further configuring the at least one processor to periodically monitor a subject's LVEF using the LVEF prediction model.

12. The apparatus of claim 11, wherein the subject is on a treatment regimen including use of a drug which increases a risk of heart failure.

13. The apparatus of claim 12, wherein periodically monitoring the subject's LVEF using the LVEF prediction model comprises comparing the subject's LVEF to a monitoring threshold value.

14. A method of left ventricular ejection fraction (LVEF) prediction, the method comprising:
using at least one processor, receiving a first electrocardiogram (ECG) datum;
using the at least one processor, inputting the first ECG datum into a LVEF prediction model comprising a masked autoencoder model, the masked autoencoder model configured to receive the first ECG datum in a modified form having masked temporal patches, reconstruct the first ECG datum including the masked temporal patches, and remove noise from the ECG datum by identifying reconstruction errors and adjusting for discrepancies, wherein the masked autoencoder model iteratively adjusts parameter values to minimize differences between reconstructed temporal patches and the masked temporal patches; and
using the at least one processor, generating a first LVEF prediction comprising a confidence metric using the LVEF prediction model.

15. The method of claim 14, wherein the method further comprises determining a diastolic dysfunction datum if the first LVEF prediction is above 50%.

16. The method of claim 14, wherein the method further comprises determining a left ventricular dysfunction datum if the first LVEF prediction is below 50%.

17. The method of claim 14, wherein the method further comprises determining a cardiac assessment datum if the first LVEF prediction is below 45%.

18. The method of claim 14, wherein the method further comprises determining a left ventricular dysfunction datum if the first LVEF prediction is below 40%.

19. The method of claim 14, wherein the method further comprises determining a sudden death risk datum if the first LVEF prediction is below 35%.

20. The method of claim 14, wherein the method further comprises determining a left ventricular dysfunction datum if the first LVEF prediction is below 30%.

21. The method of claim 14, wherein the method further comprises:
using the at least one processor, receiving a second electrocardiogram (ECG) datum recorded after the first ECG datum;
using the at least one processor, inputting the second ECG datum into the LVEF prediction model;
using the at least one processor, receiving as an output from the LVEF prediction model a second LVEF prediction; and
using the at least a processor, determining a LVEF decline datum if the second LVEF prediction is at least 5% lower than the first LVEF prediction.

22. The method of claim 14, wherein the method further comprises:
using the at least one processor, receiving a second electrocardiogram (ECG) datum recorded after the first ECG datum;
using the at least one processor, inputting the second ECG datum into the LVEF prediction model;
using the at least one processor, receiving as an output from the LVEF prediction model a second LVEF prediction; and
using the at least one processor, determining a LVEF decline datum if the second LVEF prediction is at least 10% lower than the first LVEF prediction, wherein the 10% decline threshold corresponds to a monitoring threshold value associated with patient drug regimens including mavacamten and heart failure treatment drugs.

23. (canceled)

24. The method of claim 14, wherein the method further comprises periodically monitoring a subject's LVEF using the LVEF prediction model.

25. The method of claim 24, wherein the subject is on a treatment regimen including use of a drug which increases a risk of heart failure.

26. The method of claim 25, wherein periodically monitoring the subject's LVEF using the LVEF prediction model comprises comparing the subject's LVEF to a monitoring threshold value.
